(19) United States
(12) Patent Application Publication
(10) Pub. No.: US 2008/0082087 A1
(43) Pub. Date: Apr. 3, 2008

(54) Fast panretinal laser photocoagulation

(76) Inventors: Elliott S. Eisenberg, San Francisco, CA (US); Tony Partono, San Francisco, CA (US)

Correspondence Address: Elliott S. Eisenberg, 1215 Greenwich #4A, San Francisco, CA, 94109, US

(21) Appl. No.: 11/523,437

(22) Filed: Sep. 19, 2006

(51) Int. Cl. A61B 18/18

(52) U.S. Cl. 606/4

(57) ABSTRACT

A laser beam deflection device is disclosed for automating panretinal photocoagulation (PRP). Used in ocular surgery with lasers the apparatus consists of an external casing (14) which is placed in the laser pathway of a delivery system. Internally, a central rotary reflecting mirror (2) mounted on a controlling micromotor (4) is used to reflect incident laser energy (10). The beam's pathway is then altered after striking the central mirror and is redirected to a peripheral mirror (6) mounted on disc (8). After hitting the second mirror a further redirection of the laser pulse occurs. Each time the central mirror (2) rotates a fixed angular extent the laser energy (10) is driven to a different peripheral mirror (6). Repetitive bursts of the laser are timed with the circumferential motion of the central rotary mirror via a control box (18). The circular arrangement of peripheral mirrors (6) in coordination with the rotating central mirror (2) results in a ring of laser shots for delivery to an appropriate fundus contact lens. This results in greater speed, accuracy, and efficiency in performing panretinal laser photocoagulation.

### CROSS-REFERENCE TO RELATED APPLICATIONS

This application is related to application Ser. No. US11/024,308, Filed 2004 Dec. 28 by one of the present inventors and Ser. No. US11/193,735, Filed 2005 Jul. 29 by both of the current inventors.

### FEDERALLY SPONSORED RESEARCH

Not applicable

### SEQUENCE LISTING OR PROGRAM

Not Applicable

### BACKGROUND OF THE INVENTION

1. Field of the Invention

The current invention relates to ophthalmic devices which use laser energy to deliver therapeutic treatment to the posterior segment of the eye.

2. Prior Art

The incidence of diabetes mellitus and its complications are rapidly increasing. A study released by the Centers For Disease Control and Prevention (CDC) showed that the disease rose 14% in the last two years. A prevalence estimate in 2002 showed that 6.3% of the US population, approximately 18.2 million people, carried the diagnosis. Current studies now show 20.8 million Americans are afflicted. Worldwide projections expect the incidence of diabetes to increase from 171 million in the year 2000 to 366 million by 2030.

Among the microvascular complications of diabetes mellitus diabetic retinopathy has a debilitating impact. It eventually develops in almost all people that live long enough with the diagnosis. In advanced countries diabetic retinopathy is a leading cause of blindness in the working age population (20-74 years of age). 4.1 million US citizens over the age of 40 have diabetic retinopathy. Decreased vision and subsequent reductions in functional status are valid fears in these patients.

Although diabetic visual disease is broadly separated into the categories of ‘background’ and ‘proliferative’ both are responsible for impaired eyesight. Proliferative diabetic retinopathy is the most severe form of the ocular pathology. In the 1970's a large clinical trial (Diabetic Retinopathy Study) demonstrated that panretinal laser photocoagulation (PRP) with laser light could reduce visual loss by approximately 50%. Although the mechanisms for this salutary effect are still subject to question this style of laser treatment has been practiced for over 35 years. Furthermore, PRP is occasionally also used for severe background diabetic retinopathy when patient follow up is poor for geographic or social reasons. Physicians are given some latitude in instituting this treatment.

Panretinal photocoagulation also has a role in treating disorders other than diabetic retinopathy. Many diseases such as branch retinal vein occlusion with disc neovascularization, central retinal vein occlusion with neovascular proliferation, and rubeotic glaucoma are also targets for panretinal photocoagulation.

There are currently three delivery formats for effectuating this type of ablation (PRP). One of them, the endoprobe, requires an operative setting and a vitrectomy. Thus, it is the least utilized methodology to deliver this treatment. The other two delivery techniques involve a slit lamp biomicroscope in conjuction with a fundus contact lens or a laser indirect ophthalmoscope with the aid of a hand held condensing lens. The latter two scenarios are outpatient procedures and are used to institute the majority of treatments of panretinal photocoagulation (PRP). All the delivery formats require careful aiming, a high degree of operator dexterity, and a considerable time investment. Most authorities advocate that at least 1500-2000 laser burns (500 micron size) be placed in the posterior segment of the eye. Except in the cases where retrobulbar anesthesia is used the treatments are often painful. The standard of care for the operation often requires separate sessions (multiple appointments) to deliver therapy. In fact, the Early Treatment Diabetic Retinopathy Study recommended that no greater than 900 laser applications be applied at any one operative appointment. Not uncommonly three treatment sessions are required to complete an initial course of therapy. Thus, the total treatment time may require 60 minutes. And, if the disease does not respond or regress more laser applications are typically completed. Since the posterior anatomic pole of the eye is spared from treatment (ie the macular area and the optic nerve) the net result of treatment is a ring of laser ablations placed in a grid fashion in the back of the eye. Until recently treating doctors have had to individually trigger each laser application separately. This is accomplished in all delivery formats by stepping on a foot pedal. To speed treatment some newer technologies have provided a burst of multiple laser applications with a single depression of a foot switch.

Since the endoprobe delivery format requires an operating room setting the slit lamp laser delivery system is probably the most common technique for worldwide PRP administration. In this scenario a laser source is connected to a biomicroscope through an optical cable. Holding a fundus contact lens on the patient's anesthetized eye the examiner focuses an attenuated beam on the area of the retina that is desired for ablation. After setting laser therapy parameters such as power, beam size, treatment duration, and wavelength the doctor fires the laser by activating a foot switch. Usually the burns are placed in grid pattern with approximately 500 microns of separation between applications. Significant coordination is required by the treating physician since he/she must steady the contact lens on the patient's eye, rotate the device, aim the laser beam, and trigger the shots manually. With only a few exceptions the laser burns are placed one shot at a time. The process can be tedious and time consuming for both the doctor and patient.

Alternatively the binocular indirect ophthalmoscope used with a hand held condensing lens can be used to effectuate PRP. Again a laser source is connected via an optical cable to the delivery device—in this case a headpiece worn by the examiner. Using a separate illumination beam running through the head gear the physician is able to view the ocular posterior segment while hand holding a lens in front of the patient's eye. Activating a foot switch causes laser light to penetrate the globe in the desired location chosen by the treating surgeon. Small spatial variations in the position of the lens, the eye, or the physician headpiece can blur the view or cause inadvertent treatment errors in the wrong location. Coordination for successful completion of the procedure is mandatory. Like in most other delivery formats the treatments are applied individually—one at a time.

A device to speed the procedure, a device to reduce operator coordination, a device that would decrease patient pain, and a device that would minimize aiming errors would be desirable. The prior art has addressed a few of these objectives. Bahmanyar et al (2000) in U.S. Pat. No. 6,066,128 proposed an optical mechanism to cut treatment time. Using a microlens array and a collimating lens they were able to split a single laser beam into four simultaneous discharges. Thus the authors proposed that a single depression of an activating switch would result in four treatment burns. However, their invention did not address or reduce issues such as aiming errors, operator coordination, or patient pain. Since the treating surgeon would still be forced to manually activate applications one at a time the overall operation would remain tedious to perform. In U.S. patent application Ser. No. 11/193/735 Eisenberg and Partono (2005) successfully addressed issues of speeding PRP, decreasing operator errors, cutting complications, and reducing patient pain. Their device, when interposed in a delivery apparatus between the laser source and the patient's eye, redirects the treatment light. Acting as a laser beam path diverter the invention alters the energy path so that treatment applications are placed in a circumferential pattern. Using a disc mounted with mirrors or prisms a micromotor with attached gearing drives a plate with rotational force. Timed and synchronized with laser firing the tool, used with the appropriate fundus contact lens, results in automating the process of panretinal photocoagulation. The efficacy, viability, stability, and functionality of their technique remain unchallenged. However, the authors current invention, approached from a mechanistically different viewpoint, will reduce the cost of building the apparatus. The net result will continue to remain the automation of panretinal photocoagulation. Recently the problem of reducing the speed of the procedure has been broached by OptiMedia Corp—a Santa Clara, Calif. based company. As of this writing their patents are pending for a for a laser delivery system named PASCAL. Basically, a pattern scan laser is designed to treat retinal disease by applying a predetermined pattern or grid of applications after a single triggering of the instrument. A proprietary pattern generation method employs short laser pulses in rapid sequence. In one scenario the single tap of a foot peddle switch can cause a scanning laser to execute 25 treatment spots of laser light. However, their machine, while laudably reducing PRP treatment times has a number of problems that will be ameliorated by the current invention. First, the scanning laser is part of a platform integrated with a sophisticated computer scanning system that is expensive (anticipated price of >$75,000). Without purchasing the entire package many end users will not be able to afford and marshal the technology. Second, the pulse duration of the scanning system is shorter than other currently used lasers. While this may decrease patient pain with PRP it will preclude ubiquitous usage with older conventional laser photo coagulators currently in operation. Finally, in considering the delivery technique for the PASCAL system the apparatus does not reduce user coordination and does not address aiming errors.

3. Objects and Advantages

Several objects and advantages of our invention are:
a) to provide greater speed in the treatment of panretinal laser photocoagulation;
b) to provide a device that minimizes user coordination;
c) to provide an apparatus that reduces operator aiming errors;
d) to provide an instrument that decreases patient pain in PRP;
e) to provide an adaptation that diminishes complications of laser surgery;
f) to provide a methodology of laser photocoagulation that is safer;
g) to provide an article of manufacture that automates the delivery of panretinal photocoagulation.

### Additional goals and advantages of our invention will become apparent from an evaluation of the drawings and the following description.

### SUMMARY

The present invention is a device and method for partially automating the therapeutic treatment of laser panretinal photocoagulation. It consists of a body of external housing, a rotating central micromotor with a mirror or prism mounted on its surface, a disc containing a circumferential arrangement of reflective mirrors/prisms so as to redirect laser beam energy, a mechanism for attaching said instrument within a laser delivery system, and a control box.

### DRAWINGS—FIGURES

FIG. 1 shows a view from the top looking down on an adaptor with a central mirror installed on a rotating micromotor and peripherally a ring of contiguous mirrors lying on a disc.

FIG. 2 shows a lateral view with a laser beam striking the central mirror, subsequent reflection, and then redirection to a peripheral mirror.

FIG. 3 shows a view from the bottom of the invention depicting the external housing plus threaded attachment screw and a central mirror mounted to the micromotor.

FIG. 4 shows an exploded view of the machine.

FIG. 5 shows the external housing of the device with threaded attachment screw.

FIG. 6 shows the elements of the device connected to a control box.

### DRAWINGS—REFERENCE NUMBERS

2 central mirror/prism

4 rotating micromotor

6 peripheral mirror

8 mounting plate

10 entering laser beam

12 reflected exiting laser beam

14 external housing

16 threaded screw attachment mechanism

18 housing unit attached to control box with cable

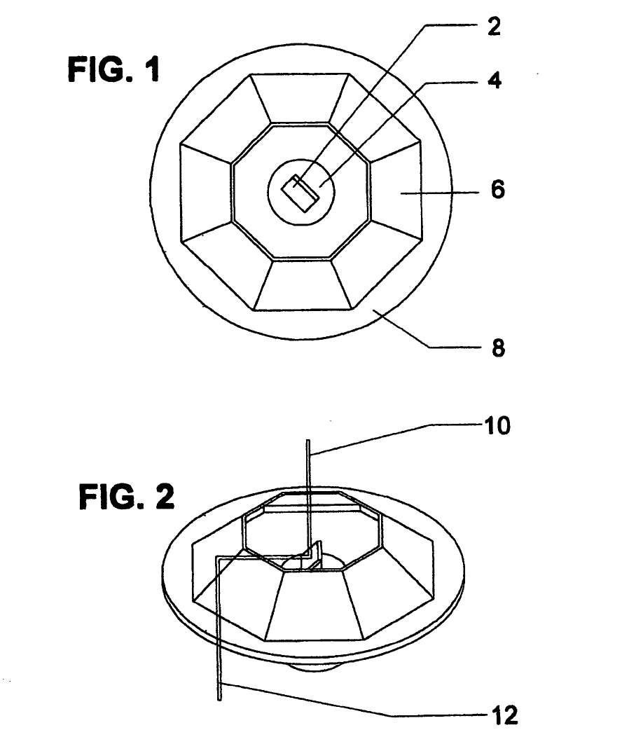

### DETAILED DESCRIPTION—PREFERRED EMBODIMENT FIGS. 1-6

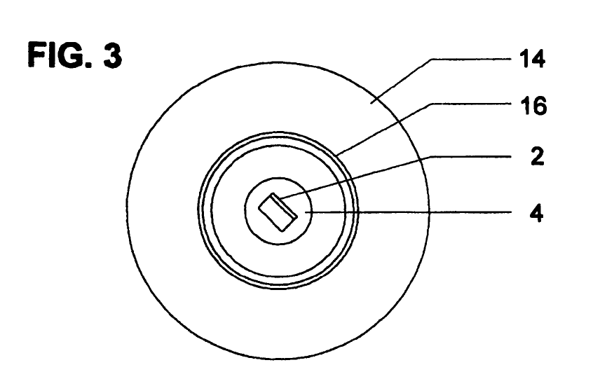

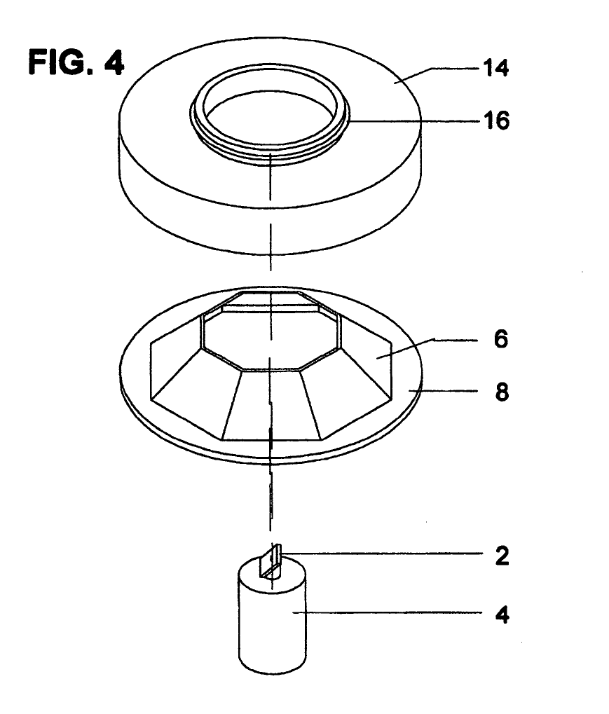

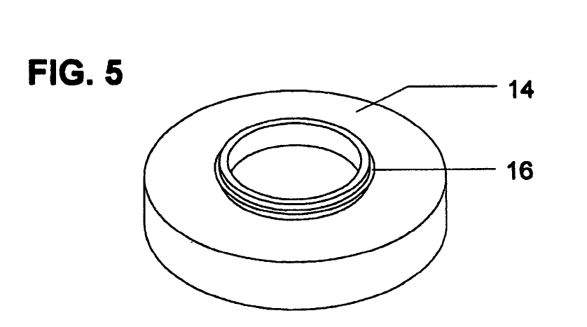

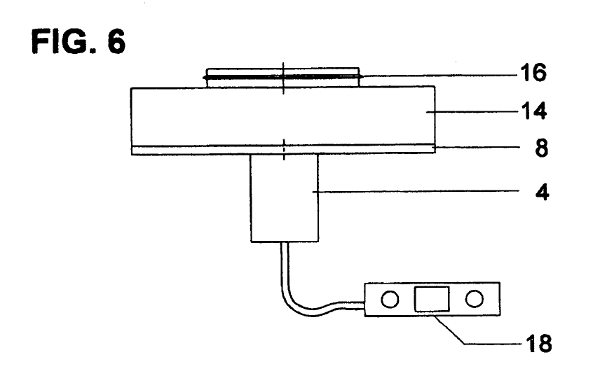

Preferred embodiment of the invention is shown in FIGS. 1-6. The main components of the laser adaptor are seen in the top view of FIG. 1. A supporting plate or disc 8 is used to mount a ring of peripheral mirrors 6. Centrally a single mirror or prism 2 is attached to the top of a rotating micromotor assembly 4. FIG. 2 depicts an oblique lateral view of the device with incident laser beam 10. As the beam strikes the centrally mounted mirror it is deflected peripherally and subsequently exits the system as displaced laser beam 12. FIG. 3 images the device from below with micromotor 4 seen with mirror 2 mounted on its superior end. External housing 14 provides a casing for the instrument. An exploded view of the deflecting adaptor is seen in FIG. 4. The top drawing shows the external housing 14 of the instrument. The middle drawing views a ring of circumferential mirrors 6 supported on disc 8. And the bottom drawing isolates rotating cylindrical micromotor 4 with mounted central mirror 2 on its top surface. FIG. 5 contains the external housing of the adaptor 14 with a threaded screw 16 attachment mechanism. A cable with the device connected to a control box 18 is seen in FIG. 6.

### OPERATION-PREFERRED EMBODIMENT—FIGS. 1,2,4,6

This invention which alters the usual pathway of laser light is consistent in operation with known methodologies currently established in performing panretinal photocoagulation. A slit lamp biomicroscope is most often the delivery format for most treatments. It can, however, be used in other laser delivery systems such as the indirect ophthalmoscope or with the ocular endoprobe. Using topical anesthesia on the patient's cornea the physician applies a fundus contact lens on the treatment eye along with a coupling agent. With the preferred embodiment a special contact lens as delineated by Eisenberg (2004) in U.S. patent application Ser. No. 11/0241,308 will be chosen. This will facilitate a ring of laser photocoagulation applied to the posterior segment of the eye. The physician will choose the correct contact lens to treat either mid peripheral or the peripheral retina depending on the clinical circustances. In a procedure well known in the art the operator then sets variable laser parameters such as duration, power, wavelength, and spot size. A regulator and control box associated with this invention will facilitate the optimal settings along with the spot position (FIG. 6). For maximal efficiency in administering treatments it is anticipated a broad beam laser will be chosen. Nothing, however, prevents the device from being used with any of the current spot sizes of laser wavelengths employed in standard usage at the time of this application. Furthermore, nothing prevents the device from being used, now or in the future, for any wavelength in the electromagnetic spectrum that is found to be beneficial for panretinal photocoagulation. After the examiner checks the focus on the contact lens it is anticipated the laser adaptor can be placed in the beam pathway if it has not already been done. At that juncture activating a trigger switch will send a laser pulse centrally to the mirror 2 (FIG. 1) mounted on the surface of the device's micromotor 4 (FIG. 4). The laser energy will then be deflected to a peripheral mirror 6 (FIG. 1). Again reflection will take place and the beam 12 (FIG. 2) will exit the adaptor headed toward the appropriate mirror on the ring fundus contact lens by Eisenberg (2004). Henceforth, the same beam will again be redirected and enter the patient's eye to the correct anatomic zone within the retina. After the completion of an initial laser pulse and its subsequent deflection the central mirror on the device will rotate on command driven by the micromotor. A fixed amount of rotation will align the central mirror accurately with the next closest peripheral mirror mounted on disc 8 (FIG. 1). As the laser again fires delivering energy to the central mirror the new beam will be deflected to a different peripheral mirror than with the original shot. Its subsequent redirection will exit the system to a new position on a corresponding fundus contact lens mirror. The process will be repeated in sequence. First, the laser will send a pulse to the central rotating mirror. The beam will then be reflected to a peripheral mirror and redirected. Then the central mirror will rotate a fixed angular extent and another pulse will be sent to a different peripheral mirror. It too will undergo redirection. Automatically the laser will pulse in conjunction with central mirror rotation such that all mirrors arranged on a circle on the device will be hit with laser energy. They will then redirect the beams of light to the corresponding mirrors within a fundus contact lens on the eye. In this fashion a ring of laser spots will be delivered to the interior of the eye. It is anticipated that an operator triggering the first laser shot will initiate an automatic sequence of spots applied circumferentially by the device. After a ring of treatment is applied the surgeon can decide whether the same process should be repeated in a new area of the retina. At that point simply exchanging the contact lens on the patient's eye with different mirror angulations will result in further treatments delivered to alternative posterior segment locations. The sequential firing of the laser timed with a device which diverts laser light in a circular pattern will produce a faster delivery of panretinal laser photocoagulation. Furthermore, it will reduce aiming errors and the coordination necessary to perform this treatment. And it will obviate the necessity for a treating physician to manually aim and trigger the laser one shot at a time.

### DESCRIPTION—ALTERNATIVE EMBODIMENTS

A number of alternatives exist for different embodiments to this invention. Many involve the method for attaching the device to a laser delivery system. While the preferred embodiment in the current scenario favors a thread/screw mechanism of attachment to a laser apparatus other ways exist. The instrument could be secured and aligned within a delivery system by a clip on mechanism which would allow the assembly to slide in and out of position. In addition this laser adaptor could pivot or rotate on a fixed axis as a means to fasten it to a laser platform. Furthermore, the deflector might be attached to a mobile or rotating arm which could swing in and out of position within a therapeutic laser assembly. However, the method of securing the device within a system is not key to the spirit of the invention. It remains ancillary to the central thesis.

Alternatively the control regulator for the device could be designed with a multitude of possibilities. Instead of a cable connection to a control box the device might have wireless regulation. The laser control panel might be integrated with the device electrically so as to modulate the micromotor in conjunction with laser pulses. Somewhat analogous to the methodology of attaching the device, the control of the system is secondary to the main concept of the invention.

Finally, one skilled in the art might devise an instrument paralleling the functional operation of this beam deflector. This could be accomplished by arranging multiple laser beams around a central axis. Each could be designed to fire in sequence. Or they might fire simultaneously. The net effect would produce a ring of photocoagulation similar to the current device. Alternatively, laser beam splitters or a lens array might be interposed within a laser delivery system. In this fashion a single laser beam would be transformed into multiple beams. If geographically arranged in space to produce a ring of panretinal therapy this would mimic the current invention.

### ADVANTAGES

It can be ascertained from our description that our invention has a number of advantages:

a) The time to complete panretinal photocoagulation will be shortened. Triggering the automatic rotation of a central mirror that will deflect the laser light peripherally and subsequently into an appropriate fundus contact lens will effectuate a faster delivery.

b) This apparatus will decrease user coordination since the operator will not have to manually aim and fire the laser one shot at a time.

c) Physician aiming errors will be diminished since the device will facilitate an automated delivery of pulses to the interior of the eye.

d) The methodology is likely to reduce patient pain associated with panretinal photocoagulation. If used in conjunction with a broad beam laser the invention may deliver less total energy to the retina/choroid complex. In addition the instrument will work well with short pulse durations.

e) Patient safety will be enhanced with this apparatus since a faster procedure will result in less physician fatigue. Also, an automated process will prevent misdirected laser energy due to operator errors.

f) The complications of laser surgery in PRP will be reduced. When less energy penetrates the eye adverse events such as visual field contraction, contrast sensitivity reduction, and nyctalopia will be diminished.

g) The machine will partially automate a treatment process which is heavily laden with manual input.

h) The device will be cheaper to produce and hence more ubiquitously available for world usage than the prior art.

### CONCLUSIONS, RAMIFICATIONS, SCOPE

The reader can see that partially automating the process of panretinal photocoagulation by this invention will make it a safer, a faster, and a more efficient treatment. This is accomplished by mechanically diverting and transforming a laser beam into a circular configuration. In addition, the laser pattern is triggered sequentially to perform repetitive firing. In conjunction with an appropriate fundus contact lens the net effect will be to provide a ring of laser photocoagulation to the interior of the eye. This will reduce treatment times, operator errors, patient pain, complications, and the coordination required to perform the procedure.

Although the above description has many specificities these should not be construed as limitations on the scope of the invention. Instead they should be viewed as variants of the preferred embodiment. Other options are possible in addition to the one described. For example, the number and shapes of the peripheral mirrors of the device are variable. And the mechanism of attaching the instrument to a laser delivery system has many alternatives. Finally, the control and regulation of the invention within a laser platform has a multitude of options.

Thus the scope of the invention should be determined by the appended claims and their legal equivalents.

We claim

1. A laser beam adapting device for automating panretinal laser comprising:
a) a solid stationary disc or ring mounted with peripheral reflecting mirrors or prisms to redirect a laser beam,
b) a micromotor mounted with a central rotating mirror designed to fit within said solid disc and translate rotational force to to said mirror thus coordinating sequential laser light reflection to peripheral mirrors,
c) a control apparatus to time the repetitive firing of laser pulses with the circular rotations of said central mirror, whereby when placed within a laser delivery system the adaptor will function to fire a circular pattern of laser applications.

2. The beam diverting device in claim 1 wherein the micromotor and solid stationary disc are enclosed with a metallic body casing.

3. The beam diverting device in claim 1 wherein the casing has a screw thread attachment to a laser delivery system.

4. The beam diverting device in claim 1 wherein the attachment mechanism is a mobile mechanical arm.

5. The beam diverting device in claim 1 wherein the mechanism of attachment is by a clip-on or sliding arrangement.

6. The beam diverting device in claim 1 wherein the micromotor is attached to a control box via a cable.

7. The beam diverting device in claim 1 wherein the micromotor is regulated by a wireless connection to a control box.

8. The beam diverting device in claim 1 wherein the reflective surfaces in the device are prisms.

9. A laser work station apparatus comprising:
(a) a body and means of attachment to a laser delivery system,
(b) a central micromotor mounted with a reflective rotating mirror or prism to alter the pathway of a laser beam,
(c) a solid stationary plate or ring mounted with a circular arrangement of peripheral mirrors for redirecting laser energy,
(d) a control system to coordinate laser shots sequentially with fixed rotations of said central mirror, whereby triggering the system will produce an automatic ring of laser pulses.

10. The laser work station in claim 9 wherein the functional elements are enveloped in a casing.

11. The laser work station in claim 9 wherein the attachment mechanism is a clip-on device.

12. The laser work station in claim 9 wherein the motor is controlled remotely in a control box.

13. The laser work station in claim 9 wherein the attachment method is a mobile rotating arm.

14. The method of automatically producing a ring of laser treatment comprising the steps of:
(a) deflecting a beam path by a central rotary mirror,
(b) moving the central mirror with an attached micromotor,
(c) redirecting laser pulses via peripheral mirrors,
(d) coordinating sequential timed laser pulses with specific angular displacements in said central mirror whereby the laser will fire an circular pattern of applications with a single triggering.

15. The method of claim 14 wherein said micromotor is linked to a control box.

16. The method of claim 14 wherein the device slides in and out of position with a clip-on attachment.

17. The method of claim 14 wherein the system is enclosed in a metallic casing.

18. The method of claim 14 wherein the assembly is moved into position on a mechanical arm.

19. The method of claim 14 wherein the instrument is screwed into place within a laser delivery system.
